(19) United States
(12) Patent Application Publication
(10) Pub. No.: US 2007/0027341 A1
(43) Pub. Date: Feb. 1, 2007

(54) Process and catalyst for the preparation of aldonic acids

(76) Inventors: Michele Rossi, Peschiera Borromeo (IT); Serena Biella, Milano (IT); Massimiliano Comotti, Trezzo Sull'Adda (IT)

Correspondence Address: YOUNG & THOMPSON, 745 SOUTH 23RD STREET, 2ND FLOOR, ARLINGTON, VA, 22202, US

(21) Appl. No.: 10/562,838

(22) PCT Filed: Jun. 22, 2004
(86) PCT No.: PCT/IB04/02059

§ 371 Date: May 15, 2006

(30) Foreign Application Priority Data
Jul. 1, 2003 (IT) M12003A001348
Oct. 21, 2003 (IT) M12003A002043

(51) Int. Cl. B01J 21/18; C07C 51/16

(52) U.S. Cl. 562/525; 502/182; 502/184; 502/185; 977/896

(57) ABSTRACT

This invention relates to a process for the preparation of aldonic acids (in particular gluconic acid) by oxidation of aldoses with oxygen or with a gas containing oxygen, in the presence of supported bimetallic catalysts based on gold and platinum.

### FIELD OF INVENTION

This invention relates to a process for the preparation of aldonic acids, and in particular to a process for the preparation of aldonic acids by catalytic oxidation of aldoses, and catalysts suitable for said purpose.

### PRIOR ART

Aldonic acids, with the general formula (I)
HOCH₂—(CHOH)ₙ—COOH (I)
(where n is an integer between 1 and 4), in particular gluconic acid, are important industrial products used as water-soluble cleaning agents or additives for foods and drinks. Gluconic acid is manufactured on an industrial scale by fermentation of glucose or raw materials containing glucose. The problems involved in these processes are the difficulty of separating bacteria, moulds and enzymes, the formation of by-products, and the difficulty of eliminating waste water.

For these reasons, methods for the preparation of aldonic acids by catalytic oxidation of aldoses were recently studied and perfected. However, these methods are only applicable on an industrial scale if the catalyst has excellent activity, selectivity, productivity and duration.

Heterogeneous catalysts for processes operating in gaseous phase or liquid phase are widely used in the petrochemical, basic chemicals, fine chemicals and pharmaceutical chemicals industries. Many of them are constituted by catalytically active metal particles, dispersed on a support which has the function of anchoring them and preventing their aggregation, thus ensuring a high specific area of metal. These catalytic systems are prepared by contact between the metal precursor and the support, operating in accordance with long-established experimental methods, such as absorption, precipitation, impregnation, vapour deposition and colloidal deposition.

Catalysts known for the oxidation of aldoses include noble metals dispersed on porous supports. Catalysts based on metals in the platinum group are described as very efficient (see, for example, U.S. Pat. Nos. 5,132,452 and 4,843,173), especially when they are modified with bismuth. A very active catalytic system consists of a combination of palladium, platinum and bismuth supported on carbon. The drawback of these catalysts is that oxidation takes place in an alkaline medium: the product of the reaction is therefore not the free acid or the corresponding lactone, but the corresponding salt of an alkaline metal or other metal (such as a calcium salt). Moreover, isomerisation of carbohydrates is observed at high pH levels, and the duration of the catalyst is limited.

It has recently been found (S. Biella, G. L. Castiglioni, C. Fumagalli, L. Prati and M. Rossi, Catalysis Today, 72, 43-49 (2002)) that a supported gold catalyst enables aldoses with the general formula (II)
HOCH₂—(CHOH)ₙ—CHO (II)
(where n is an integer between 1 and 4) to be oxidised to aldonic acids (I) in an aqueous support in the presence of oxygen or a gas containing oxygen (such as air or oxygen-enriched air) under bland conditions, ie. at relatively low temperatures and pressures. One of the characteristics of gold-based catalysts is that they are active in both basic and acid environments. Both aldonic acid salts and free acids in balance with the corresponding lactones can therefore be obtained. In particular, in the case of glucose, both gluconic acid salts and free gluconic acid in balance with its lactonised forms can be obtained, as described in Italian patent n. 01313696 of 16 Dec. 1999 filed by Lonza. However, oxidation of glucose with the gold catalyst in the absence of a base does not produce yields comparable with oxidation in an alkaline environment or with enzymatic systems, due to the low activity of the catalyst in a non-basic environment.

The problem therefore arises of devising a catalyst for oxidation of aldoses to aldonic acids which has a high level of activity, is easy to recycle, and can operate under bland conditions, regardless of the pH.

### DESCRIPTION OF THE INVENTION

It has now been discovered that bimetallic catalysts based on gold and platinum are far more active and selective than monometallic catalysts based on gold.

This invention relates to a process for the production of aldonic acids, their salts or lactones, with the general formula (I), comprising oxidation of an aqueous solution of aldoses with formula (II) with molecular oxygen or a gas containing oxygen, at a temperature of between 30 and 90° C., without pH control, in the presence of a catalyst based on metallic gold and metallic platinum. The aqueous solution preferably contains between 3 and 50% in weight of aldose with formula (II), and the partial oxygen pressure is preferably between 0.2 and 10 bars.

The catalyst is formed by metallic gold and metallic platinum or alloys thereof, deposited in a highly dispersed form on an organic or inorganic support. The total metal content of the supported catalyst is between 0.1 and 10% in weight, preferably between 0.5 and 2% in weight, and even more preferably the total metal content is 1% in weight. The weight ratio between gold and platinum is between 5 and 0.2, preferably between 3 and 0.5, and is even more preferably 2. The metal particles preferably have a diameter of between 1 and 20 nanometres, in particular between 3 and 7 nanometres. The preferred support is activated carbon with a specific surface of at least 200 m²/g, but titania, alumina or other inorganic or organic supports can also be used. According to a particularly preferred embodiment of the invention, the support is activated carbon with an average particle size of 5 to 100 micrometres, and a specific surface of 1200 m²/g.

The catalyst can be manufactured with the conventional techniques specified above. Preferably, however, the catalyst is obtained by a process comprising deposition of metals on the chosen support, from a colloidal solution of gold and platinum salts or acids, with reducing agents such as formaldehyde, sodium tetrahydroborate, ammonium or hydrogen citrate, in the presence of colloid-protecting agents such as polyvinyl alcohol, tetrahydroxymethylphosphonium chloride, or the like. Said process constitutes a further subject of this invention.

It has also surprisingly been found, and this finding constitutes a further subject of the invention, that a bimetallic catalyst according to this invention which is particularly active, and possesses other advantages, can be obtained with the process described above but using polyhydroxylated molecules as colloid protectors (or stabilisers), especially a monosaccharide with six carbon atoms or a disaccharide whose monosaccharide units contain six carbon atoms, in particular glucose, fructose or saccharose. The catalyst thus obtained is not only far more active than one prepared using the protectors specified above, conditions being equal, but also presents considerable operational advantages. Firstly, the preparation procedure for the catalyst obtained with known protecting agents requires filtration and thorough washing of the catalyst after addition of the support in order to eliminate said agents, which would otherwise pollute the catalyst, inhibiting its activity, with the risk of poisoning if the acids obtained are destined for the food industry, for example.

However, the catalyst obtained in the presence of polyhydroxylated compounds can be isolated by filtration and used as it is, without being washed.

Even more simply, addition of the support and subsequent filtration can be omitted, and the aldose to be oxidised is added directly to the colloidal suspension of the catalyst.

In the process according to the invention for the preparation of aldonic acids, the weight ratio between catalyst and aldose with formula (II) can be less than 0.01, but is preferably between 0.05 and 0.30.

The process to which the invention relates therefore allows the preparation of glyceric acid (n=1), erythronic and threonic acid (n=2), ribonic, xylonic, lyxonic and arabinonic acid (n=3) and hexonic acids (n=4), such as gluconic, galactonic or mannonic acid. As stated, the process is particularly suitable for the preparation of hexonic acids; preferably for the production of gluconic acid from glucose, and in particular for the production of D-gluconic acid from D-glucose.

This invention offers the following advantages over the use of catalysts based on platinum and/or palladium or monometallic gold catalysts:
high activity and productivity at low temperatures and low pH values;
long life of catalyst in a pH range (2-7) which prevents isomerisation of carbohydrates;
high resistance of the catalyst to deactivation by products of oxidation or hyperoxidation;
easy recycling of depleted catalyst by combustion of carbon and recovery of noble metals.

As these catalysts can also be used at low pH values, free acids or their lactones can be directly obtained. If salts of aldonic acids are required, said acids can be treated with a suitable base.

### DETAILED DESCRIPTION OF THE INVENTION

According to a preferred embodiment, the process according to the invention comprises the following steps:
a) reduction of gold(III) and platinum(II) compounds to colloidal gold and platinum from an aqueous solution containing a gold(III) compound or a mixture of gold(III) and platinum(II) compounds and a monosaccharide with six carbon atoms or a disaccharide formed by two monosaccharide units with six carbon atoms;
b) addition of an aldose with the formula (II)
   HOCH₂—(CHOH)ₙ—CHO (II)
   wherein n is as previously defined;
c) oxidation with oxygen or a gas containing oxygen.

The solution used in step a) is prepared by dissolving an Au(III) compound, preferably chloroauric acid, or an Au(III) compound and a Pt(II) compound, preferably potassium tetrachloroplatinate, in water, so that the final concentration is 0.01-0.02 mg/ml.

The mono- or disaccharide is then added in such a quantity that the final concentration is between 0.1 and 30 mg/ml, and preferably 30 mg/ml. The preferred monosaccharides are glucose and fructose, and the preferred disaccharide is saccharose.

The reduction is performed with a reducing agent selected from among sodium borohydride, formaldehyde, formic acid and salts thereof, citric acid and salts thereof, where the term “salts” preferably indicates sodium, potassium, ammonium or hydrogen salts. NaBH₄ is preferably used, in the same quantity in weight as the Au(III) compound or the Au(III) and Pt(II) compounds. In this way a dispersion is obtained wherein the colloidal particles have an average diameter of between 1 and 12 nm, and more precisely between 3 and 6 nm.

A support can be added before reduction, especially if the catalyst is to be filtered and not used immediately. The preferred support is activated carbon with a specific surface of at least 200 m²/g, but titania, alumina or other inorganic or organic supports can also be used. According to a particularly preferred embodiment of the invention, the support is activated carbon having an average particle size of 5 to 100 micrometres and a specific surface of 1200 m²/g. The quantity of carbon is preferably 100 times the weight of the Au(III) compound or the total weight of the Au(III) compound and the Pt(II) compound, so as to obtain catalysts with a metal content of 1%.

The catalyst can be filtered after addition of the support, but in the preferred embodiment of the invention filtration is omitted, and step b) is immediately performed by adding the aldose to be oxidised. The quantity of aldose is such that the final concentration in the solution is between 0.5 and 1 g/ml, and preferably 1 g/ml.

The oxidation reaction is performed by bubbling gaseous oxygen or a gas containing oxygen, preferably at atmospheric pressure. The reaction temperature ranges between 20 and 90° C.

The process to which the invention relates is particularly useful for the synthesis of aldonic acids with formula (I), wherein n is 4, and preferably for oxidation of glucose to gluconic acid. In particular, the use of glucose as a colloid protector is highly advantageous when the catalyst is used specifically for oxidation of glucose to gluconic acid.

As stated, the process to which the invention relates is also advantageous in the food industry, because it overcomes the problems of toxicity and inhibition of catalysis previously mentioned, and also allows the process to be performed in homogenous phase, thus reducing the time and cost.

The following examples illustrate the invention in greater detail. All the concentrations are given by weight, unless otherwise stated.

### EXAMPLES

### Example 1

### Preparation of Catalyst

### Example 1a

66 mg of gold and 33 mg of platinum (Au/Pt=2) were dissolved in a minimal quantity of aqua regia. This solution was diluted with distilled water to adjust the total concentration of the two metals to 100 mg/l. 3.2 ml of a 2% solution of polyvinyl alcohol (molecular weight 10,000) in distilled water was added to this solution. A filtered solution of NaBH₄ (25 ml, 0.1 M) was added to reduce the gold and platinum to a colloidal form. This latter dispersion was placed in contact with 10 g of activated carbon powder having an average particle size of 5 to 100 micrometres and a specific surface of 1200 m²/g for 2-3 hours. The total absorption of Au and Pt on C was confirmed by ICP (Inductively Coupled Plasma) spectrometry analysis of the aqueous phase. The resulting solid was filtered and washed several times with water until the chloride ion in the water disappeared. The catalyst was partly dried in air to a water content of 50%.

### Example 1b

A catalyst with a total gold and platinum content of 1% was prepared, supported on carbon as in example 1a, but using an Au/Pt ratio of 4, starting with 80 mg of Au and 20 mg of Pt.

### Example 1c

A catalyst with a total gold and platinum content of 1% was prepared, supported on carbon as in example 1a, but using an Au/Pt ratio of 1, starting with 50 mg of Au and 50 mg of Pt.

### Example 1d

A catalyst with a total gold and platinum content of 1% was prepared, supported on carbon as in example 1a, but using an Au/Pt ratio of 0.5, starting with 33 mg of Au and 66 mg of Pt.

### Comparative Example 2a

1% of gold on carbon without platinum was prepared as described in example 1a, using 100 mg of Au.

### Comparative Example 2b

1% of Pt on carbon without gold was prepared as described in example 1a, using 100 mg of Pt.

### Example 3

### Preparation of Gluconic Acid

### Example 3a

Glucose (5 g) and the catalyst prepared as described in example 1a (167 mg calculated on the dry matter, equal to 3.3% in weight based on glucose) in distilled water (total volume 50 ml) were placed in a container thermostated at 70° C. and under oxygen pressure at 300 kPa, and the mixture was agitated at 600 rpm. Gluconic acid was produced during the reaction, and the pH of the liquid suspension decreased until a limit value of 2.5 was reached. Samples were taken at different times and analysed by HPLC and ¹²C-NMR. The results are set out in Table 1. The only organic acid found by NMR was gluconic acid. Fructose and glucaric acid were absent.

### Example 3b

Glucose was oxidised under the same conditions as described in example 3a, but using the catalyst described in example 1b. The results are set out in Table 1.

### Example 3c

Glucose was oxidised as described in example 3a, but using the catalyst described in example 1c. The results are set out in Table 1.

### Example 3d

Glucose was oxidised as described in example 3a, but using the catalyst described in example 1d. The results are set out in Table I.

### Example 3e

Glucose was oxidised as described in example 3a, but at the temperature of 90° instead of 70° C. The results are set out in Table 1.

TABLE 1

| | | | |
|---|---|---|---|
| Example 3a | Time (h) | 2.5 | 6.5 |
| (Au/Pt = 2, T = 70° C.) | Conversion % (moles) | 77 | 90 |
| | Selectivity % (moles) | 98 | 97 |
| Example 3b | Time (h) | 2.5 | 6.5 |
| (Au/Pt = 4, T = 70° C.) | Conversion % (moles) | 29 | 43 |
| | Selectivity % (moles) | 98 | 98 |
| Example 3c | Time (h) | 2.5 | 6.5 |
| (Au/Pt = 1, T = 70° C.) | Conversion % (moles) | 20 | 33 |
| | Selectivity % (moles) | 81 | 82 |
| Example 3d | Time (h) | 2.5 | 6.5 |
| (Au/Pt = 0.5, T = 70° C.) | Conversion % (moles) | 23 | 35 |
| | Selectivity % (moles) | 84 | 81 |
| Example 3e | Time (h) | 1 | 3 |
| (Au/Pt = 2, T = 90° C.) | Conversion % (moles) | 90 | 100 |
| | Selectivity % (moles) | 98 | 96 |

### Comparative Example 4a

Glucose was oxidised as described in example 3a, but using the catalyst containing gold only, prepared as described in comparative example 2a. The results are set out in Table 2.

### Comparative Example 4b

Glucose was oxidised as described in example 3a, but using the catalyst containing platinum only, prepared as described in comparative example 2b. The results are set out in Table 2.

### Comparative Example 4c

Glucose was oxidised as described in comparative example 4a, but at the temperature of 90° instead of 70° C. The results are set out in Table 2.

### Comparative Example 4d

Glucose was oxidised as described in comparative example 4b, but at the temperature of 90° instead of 70° C. The results are set out in Table 2.

TABLE 2

| | | | |
|---|---|---|---|
| Example 4a | Time (h) | 2.5 | 6.5 |
| (Au only, T = 70° C.) | Conversion % (moles) | 8 | 13 |
| | Selectivity % (moles) | 98 | 97 |
| Example 4b | Time (h) | 2.5 | 6.5 |
| (Pt only, T = 70° C.) | Conversion % (moles) | 8 | 11 |
| | Selectivity % (moles) | 88 | 85 |
| Example 4c | Time (h) | 1 | 3 |
| (Au only, T = 90° C.) | Conversion % (moles) | 16 | 53 |
| | Selectivity % (moles) | 98 | 96 |
| Example 4d | Time (h) | 1 | 3 |
| (Pt only, T = 90° C.) | Conversion % (moles) | 10 | 23 |
| | Selectivity % (moles) | 88 | 86 |

Tables 1 and 2 demonstrate that gold and platinum act synergically, because they show greater activity than the metals dispersed individually in the support. The examples demonstrate that this synergy can be optimised by varying the ratio between gold and platinum. In particular, the results indicate that the maximum synergy between the two metals is obtained when the Au/P weight ratio is 2.

Preparation of Monometallic Catalysts Based on Gold Supported on Carbon

### Example 5

20 mg of gold was dissolved in a minimal quantity of aqua regia, and the resulting solution (0.5 ml) was diluted with water to 200 ml. 6 g of glucose was added, and 20 mg of NaBH₄, dissolved in 10 ml of water, was added under magnetic agitation. 2 g of carbon powder was added to the reddish-brown colloidal dispersion thus obtained. Decolouration of the water was observed after filtration; the 1% Au/C catalyst thus prepared was 40% wet, and used without washing. X-ray analysis (XRPD) demonstrated the presence of particles with an average diameter of 4.4 nm.

### Example 6

The process was performed as described in example 5, using 200 mg of glucose.

### Example 7

The process was performed as described in example 5, using 20 mg of glucose.

### Example 8

The process was performed as described in example 5, using 6 g of fructose instead of glucose.

### Example 9

The process was performed as described in example 5, using 6 g of saccharose instead of glucose.

Catalysis Tests at a Controlled pH

### Example 10

Glucose (15 g) and the catalyst prepared as described in example 5 (75 mg calculated on the dry matter, equal to 0.5% in weight based on glucose) in distilled water (total volume 15 ml) were placed in a glass reactor thermostated at 50° C., the mixture was agitated at 600 rpm, and gaseous oxygen (1 l/min, at atmospheric pressure) was bubbled through it. The conversion of glucose over time was monitored with an automatic titrator at a controlled pH of 9.5 (Titrino®, Metrohm) by titrating the gluconic acid formed. The conversions of glucose to gluconic acid are set out in Table 3.

### Example 11

The process was performed as described in example 10, using the catalyst described in example 6.

### Example 12

The process was performed as described in example 10, using the catalyst described in example 7.

### Example 13

The process was performed as described in example 10, using the catalyst described in example 8.

### Example 14

The process was performed as described in example 10, using the catalyst described in example 9.

### Example 15

### Catalyst Based on Gold Prepared In Situ, and its Evaluation

The catalytic system was prepared as follows in the reactor used in example 10: a chloroauric solution containing 0.8 mg of gold and 240 mg of glucose in 15 ml of water was reduced with 0.8 mg of NaBH₄ at ambient temperature, and 80 mg of carbon was added. The mixture was then thermostated a 50° C.; 14.8 g of glucose was added, and the mixture was magnetically agitated and oxygenated. The catalytic conversion of glucose to gluconic acid was then monitored with an automatic titrator.

The results are set out in Table 3.

### Example 16

### Bimetallic Catalyst Based on Gold and Platinum, Supported on Carbon

The process was performed as described in example 5, but starting with a solution containing 14 mg of gold in the form of tetrachloroauric acid and 7 mg of platinum in the form of potassium tetrachloroplatinate.

### Example 17

### Catalysis Test at a Controlled pH with Supported Bimetallic Catalyst

The process was performed as described in example 10, using the catalyst described in example 16.

### Example 18

### Supported Bimetallic Catalyst Prepared In Situ, and its Catalytic Evaluation at a Controlled pH

The process was performed as described in example 15, using the following procedure: 0.8 mg of Au in the form of HAuCl₄ and 0.4 mg of Pt in the form of K₂PtCl₄ were reduced with 1.2 mg of NaBH₄ in the presence of 360 mg of glucose; 120 mg of carbon and 14.7 g of glucose were then added, and the reaction was triggered by oxygenating the mixture under agitation at 50° C. The results are set out in Table 3.

### Example 19

### Catalysis Test at a Controlled pH with Supported Bimetallic Catalyst

Glucose (5 g) and the catalyst described in example 16 (167 mg calculated on the dry matter) in distilled water (total volume 50 ml) were placed in a container thermostated at 90° C. and under oxygen pressure of 300 kPa. The mixture was agitated at 600 rpm. Samples were taken at different times and analysed by HPLC. The results are set out in Table 3.

### Example 20

### Supported Bimetallic Catalyst Prepared In Situ and Used at Free pH

The catalytic system was prepared as follows in the reactor used in example 19: a solution containing 1.2 mg of gold in the form of chloroauric acid, 0.6 mg of platinum in the form of potassium tetrachloroplatinate and 0.5 g of glucose in 50 ml of water was reduced at ambient temperature with 1.7 mg of NaBH₄, and 170 mg of carbon was then added. The mixture was thermostated at 90° C., 4.5 g of glucose was added, and it was placed under oxygen pressure of 300 kPa and agitated magnetically at 800 rpm. Samples were taken at different times and analysed by HPLC. The results are set out in Table 3.

### Example 21

### Unsupported Bimetallic Catalyst and Catalysis Tests at Free pH

The process was performed as described in example 20, without adding carbon. The results are set out in Table 3.

TABLE 3

| Example | Protector | Time (h) | Conversion % | D (nm, XRPD) |
|---|---|---|---|---|
| 10 | Glucose | 0.5 | 43 | 4.4 |
| 11 | Glucose | 0.5 | 33 | 10.0 |
| 12 | Glucose | 0.5 | 26 | 12.8 |
| 13 | Fructose | 0.5 | 33 | 3.8 |
| 14 | Saccharose | 0.5 | 30 | 3.3 |
| 15 | Glucose | 0.5 | 39 | n.d. |
| 17 | Glucose | 0.5 | 44 | n.d. |
| 18 | Glucose | 0.5 | 43 | n.d. |
| 19 | Glucose | 1 | 91 | n.d. |
| 20 | Glucose | 1 | 85 | n.d. |
| 21 | Glucose | 1 | 80 | n.d. |

n.d. = not determined

1. Process for the production of aldonic acids with the general formula (I): HOCH2-(CHOH)n-COOH (I) wherein n is an integer from 1 to 4, and their salts or lactones, comprising the following steps: a) reduction of gold (III) and platinum (II) compounds to colloidal gold and platinum from an aqueous solution containing a gold (III) compound or a mixture of gold (III) and platinum (II) compounds and, in the capacity of colloid-protecting agent, a monosaccharide with six carbon atoms or a disaccharide formed by two monosaccharide units with six carbon atoms; b) addition of an aldose with the formula (II) HOCH2-(CHOH)″—CHO (II) wherein n is as previously defined; c) oxidation with oxygen or a gas containing oxygen.

2. Process as claimed in claim 1, wherein the concentration of the gold (III) and platinum (II) compounds is 0.1-0.2 mg/ml.

3. Process as claimed in claim 1, wherein the Au (III) compound is HAuCl4 and the Pt (II) compound is K2PtCl4.

4. Process as claimed in any of claim 1, wherein the reducing agent is selected from among sodium borohydride, formaldehyde, formic acid and salts thereof, citric acid and salts thereof, and hydrogen.

5. Process as claimed in claim 4, wherein the reducing agent is sodium borohydride.

6. Process as claimed in claim 1, wherein the monosaccharide is glucose or fructose.

7. Process as claimed in claim 1, wherein the disaccharide is saccharose.

8. Process as claimed in claim 1, wherein the concentration of monosaccharide or disaccharide is between 0.1 and 30 mg/ml.

9. Process as claimed in claim 1, wherein the quantity of aldose with formula (II) added is such that the final concentration is between 0.5 and 2 g/ml.

10. Process as claimed in claim 9, wherein the quantity of aldose with formula (II) added is such that the final concentration is approx. 1 g/ml.

11. Process as claimed in claim 1, wherein the aldose with formula (II) is glucose.

12. Process as claimed in claim 1, wherein a support selected from among activated carbon, titania and alumina is added after reduction of the gold and platinum compounds, and the supported catalyst thus obtained is isolated before use.

13. Process as claimed in claim 12, wherein the support is activated carbon having an average particle size of between 5 and 100 micrometres and a specific surface of at least 200 m2/g.

14. Process as claimed in claim 13, wherein the activated carbon has a specific surface of 1200 m2/g.

15. Process as claimed in claim 12, wherein the total gold and platinum content of the catalyst is between 0.1 and 10% of the weight of the support.

16. Process as claimed in claim 15, wherein the total gold and platinum content of the catalyst is approximately 1% of the weight of the support.

17. Process as claimed in claim 1, wherein the weight ratio between metallic gold and platinum is between 5 and 0.2.

18. Process as claimed in claim 17, wherein the weight ratio between metallic gold and platinum is approx 2.

19. Process as claimed in claim 1, wherein the metals have an average particle size of between 1 and 20 nanometres.

20. Process as claimed in claim 1, wherein the partial oxygen pressure is between 0.2 and 10 bars.

21. Bimetallic catalyst based on gold and platinum for oxidation of aldoses to aldonic acids, in particular for oxidation of glucose to gluconic acid, supported on activated carbon, characterised by a total gold and platinum content of between 0.1 and 10% of the weight of the support and a weight ratio between gold and platinum of between 5 and 0.2, the metals having an average particle size of between 1 and 20 nanometres and the activated carbon having an average particle size of between 5 and 100 micrometres and a specific surface of approx. 1200 m2/g.

22. Catalyst as claimed in claim 21, characterised by a total gold and platinum content of approx. 1% of the weight of the support.

23. Catalyst as claimed in claim 21, characterised by a weight ratio between gold and platinum of approx 2.

24. Catalyst as claimed in claim 21, prepared by reducing an aqueous solution of gold (III) and platinum (II) compounds and a monosaccharide with six carbon atoms or a disaccharide formed by two monosaccharide units with six carbon atoms, adding a support constituted by activated carbon to the colloidal solution thus obtained, and isolating the supported catalyst thus obtained by filtration
